(19) United States
(12) Patent Application Publication
(10) Pub. No.: US 2007/0033681 A1
(43) Pub. Date: Feb. 8, 2007

(54) Device For Sectioning Specimens

(76) Inventors: Reinhard Lihl, Vienna (AT); Michael Zimmermann, Leopoldsdorf (AT)

Correspondence Address: HODGSON RUSS LLP, ONE M & T PLAZA, SUITE 2000, BUFFALO, NY, 14203-2391, US

(21) Appl. No.: 11/462,378

(22) Filed: Aug. 4, 2006

(30) Foreign Application Priority Data
Aug. 5, 2005 (DE) 10 2005 037 060.8

(51) Int. Cl. A61B 17/32

(52) U.S. Cl. 977/904

(57) ABSTRACT

A device (1) for sectioning specimens comprises a microtome or an ultramicrotome (3) for generating thin specimen pieces. A table (5) for placement of the microtome or ultramicrotome (3) is provided, a cold chamber (30) being provided that surrounds a region of the microtome or ultramicrotome (3) for generating thin specimen pieces. A glovebox (42) surrounds the microtome or ultramicrotome (3) and the cold chamber (30). The glovebox (30) stands on the table (5), and a coolant hose (37) connects a reservoir vessel (35) with coolant to the cold chamber (30). The coolant evaporating out of the cold chamber (30) fills the glovebox (42).

### CROSS REFERENCE TO RELATED APPLICATIONS

This application claims priority of German patent application no. 10 2005 037 060.8 filed Aug. 5, 2005, which is incorporated by reference herein.

### FIELD OF THE INVENTION

The invention relates to a device for sectioning specimens, in particular a microtome or an ultramicrotome.

### BACKGROUND OF THE INVENTION

Microtomes and ultramicrotomes are usually equipped with at least one knife that serves to produce thin sections of the specimen. Microtomes and/or ultramicrotomes are surrounded by a cold chamber. It is already known to place microtomes or ultramicrotomes in a glovebox (see page 128 of “Nanostructural Study of Raney-Type Nickel Catalysts,” Francois Devred, September 2004, Delft University Press, ISBN: 9040725039). The purpose previously was that materials that reacted chemically with nitrogen, or with the oxygen residues in nitrogen gas, were being investigated. The cold nitrogen gas was therefore not directed into the glovebox, but instead was guided out of the glovebox via a sealed connection. The glovebox itself was filled with ultrapure gases via a separate supply conduit. The complexity in this instance is extreme, since the glovebox must be sealed and it is necessary to use ultrapure gases and sealed airlocks.

The Leica EM UC6 (the new ultramicrotome) and the Leica EM FC6 (the new cold chamber) (brochure entitled “The new ultramicrotome for room-temperature section and cryosectioning”) possess a tank that is filled with liquid nitrogen from a reservoir vessel. Evaporation of the liquid nitrogen creates a continuous flow of dry, cold gas that is directed into the cold chamber. The homogeneous gas flow escapes from the chamber, which is open at the top. The homogeneous purging is intended to prevent the entry of moisture. Ice formation occurs, however, at the boundary layer between the upper side of the cold chamber and the laboratory environment, and can result in the deposition of ice crystals onto the specimen or the cut sections.

An ultramicrotome of the RMC company is known (see “Ultramicrotomes” catalog), having a cryobox placed onto the housing of the ultramicrotome. Devices of the RMC company of the MTX type, having CRX cold chambers, are of similar construction, so that here as well the entry of ice crystals into the cold chamber cannot be entirely prevented.

The devices of the existing art have the critical disadvantage that in the context of a cold chamber without a glovebox, purging with dry gas cannot completely prevent the formation of ice deposits. A boundary layer with the atmosphere forms at the upper side of the cold chamber, and can also be detected visually by the formation of a slight mist. Very small ice crystals form in this boundary layer, fall into the cold chamber, and collect during sectioning on the knife and also on the sections. These ice crystals interfere with subsequent observation under the electron microscope. Large areas of the section can be unusable.

An improvement in terms of ice formation on the cold chamber can furthermore be achieved with complex air-conditioning systems, by setting a very low relative humidity (e.g. 10%). It is not very pleasant for the user to work under these conditions, however, and the air-conditioning system consumes a great deal of energy and is also expensive to acquire and install.

Various embodiments of gloveboxes may be inferred, for example, from page 579 of the Cole-Parmer catalog.

### SUMMARY OF THE INVENTION

It is therefore the object of the present invention to create a device with which it is possible to create a low relative humidity in the environment of the cold chamber, in order to prevent ice contamination in the cold chamber. The intention is for the environment around the cold chamber to be produced in simple fashion.

This object is achieved, according to the present invention, by a device according to the present invention. The invention has the advantage that a device for sectioning specimens is provided. The device comprises a sectioning apparatus, namely a microtome or an ultramicrotome, for generating thin specimen pieces. A table is provided on which the microtome or ultramicrotome is placed, the microtome or ultramicrotome possessing a knife holder and a specimen holder. The knife holder and specimen holder are surrounded by a cold chamber. A glovebox surrounds the microtome or ultramicrotome and the cold chamber, and stands on the table. A coolant hose connects the reservoir vessel with coolant to the cold chamber, so that coolant flows out of the reservoir vessel into the cold chamber, and the coolant evaporating out of the cold chamber fills the glovebox. The glovebox has a rim at which the glovebox contacts the table, and no seal is provided between the rim and the table. The glovebox is fastened to the table with multiple fastening elements. The glovebox is embodied with a passthrough for cables and a passthrough for the coolant hose.

The microtome or ultramicrotome possesses a stereomicroscope for observation of the specimen, the stereomicroscope being sealed by means of an elastic bellows between the interior of the glovebox and the laboratory environment.

The glovebox is embodied with openings which are configured in such a way that a user can introduce his or her arm into the glovebox without causing a substantial leak.

The glovebox possesses an outlet valve or a defined opening, so that no substantial overpressure builds up in the interior of the glovebox, and so that a defined outflow of the evaporating coolant is possible.

The glovebox is equipped with an airlock that permits the introduction of tools and specimens into the interior of the glovebox without thereby allowing a large amount of moisture to enter the interior of the glovebox.

The glovebox can furthermore also be equipped with an airlock that permits the introduction of a cryosample holder of an electron microscope into the interior of the glovebox, and allows the cryosample holder to be loaded with sample carriers and sections.

Further advantages and advantageous embodiments of the invention may be inferred from the dependent claims, and are the subject matter of the Figures below as well as their descriptions.

### BRIEF DESCRIPTION OF THE DRAWING VIEWS

In the individual Figures:

FIG. 1 is a perspective view of a device for sectioning specimens that comprises a microtome or ultramicrotome and a cold chamber;

FIG. 2 is a perspective view of a system made up of a reservoir vessel for coolant (liquid nitrogen) and a cold chamber;

FIG. 3 is a perspective view of the device of FIG. 1 which is surrounded by a glovebox; and

FIG. 4 is another perspective view of what is depicted in FIG. 3.

### DETAILED DESCRIPTION OF THE INVENTION

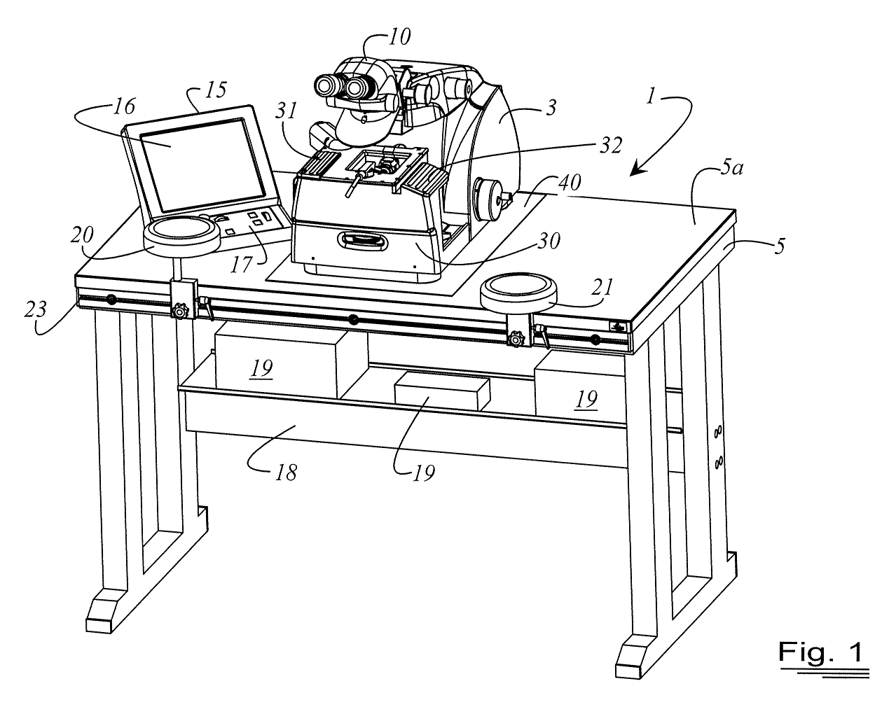

FIG. 1 is a perspective view of a device 1 for sectioning specimens. Device 1 comprises a sectioning apparatus 3 for producing thin specimen pieces. Sectioning apparatus 3 is placed onto a table 5 that defines a table surface 5a. Sectioning apparatus 3 possesses at least one knife holder and one specimen holder. Displaceable arm supports 20 and 21 are provided on table 5. Arm supports 20 and 21 are adjustable to a user's ergonomics and to a configuration of the device. Table 5 is equipped with a vibration-damping insert 40 on which sectioning apparatus 3 stands. A control unit 15 for controlling and regulating cutting apparatus 3 is additionally provided on table 5. Control unit 15 includes a display 16 and an input unit 17. Provided beneath table surface 5a of table 5 is a platform 18 on which electronics modules and power-supply modules 19 are placed.

Device 1 is made up of a sectioning apparatus 3, which may be either a microtome or an ultramicrotome, that is equipped with a cold chamber 30 that surrounds a knife holder and a specimen holder of sectioning apparatus 3. A rail 23 is mounted on table 5. Arm supports 20 and 21 are horizontally displaceable in rail 23. A stereomicroscope 10 is joined to sectioning apparatus 3 for observation of the sectioning operation by the user. Cold chamber 30 likewise comprises supports 31 and 32 for a user's hands. When a microtome or ultramicrotome is used as sectioning apparatus 3 together with a cold chamber 30, the user places his or her hands onto supports 31 and 32 on cold chamber 30, and rests his or her elbows on arm supports 20 and 21 of table 5.

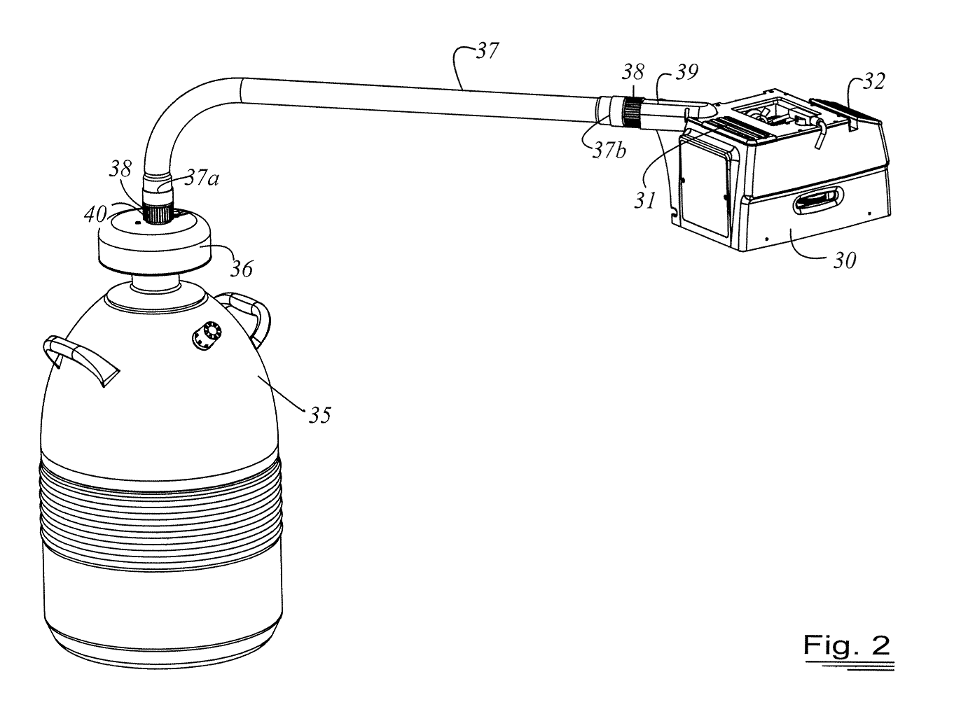

FIG. 2 is a perspective view of the connection of a reservoir vessel 35 for coolant and cold chamber 30. Cold chamber 30 is configured in such a way that it accommodates the region of the specimen holder and knife holder of a microtome or ultramicrotome (not depicted), so that a low temperature can be established. Reservoir vessel 35 is a Dewar. A pump 36 that projects into the Dewar or reservoir vessel 35 sits on reservoir vessel 35. A coolant hose 7 connects reservoir vessel 35 to cold chamber 30. Coolant is transported out of reservoir vessel 35 through coolant hose 37 to cold chamber 30. Cold chamber 30 comprises supports 31 and 32 for a user's hands when the user is operating the microtome or ultramicrotome equipped with cold chamber 30. Coolant hose 37 is made up of a transport hose and an insulating sheath. Coolant hose 37 comprises a first end 37a and a second end 37b. First and second ends 37a, 37b of coolant hose 37 are equipped with a rotatable and thermally insulating connecting element 38. The thermally insulating connecting element 38 is identical at the first and at the second end 37a, 37b. Connecting element 38 coacts with a corresponding connecting element 39 on cold chamber 30, and with a corresponding connecting element 40 on reservoir vessel 35.

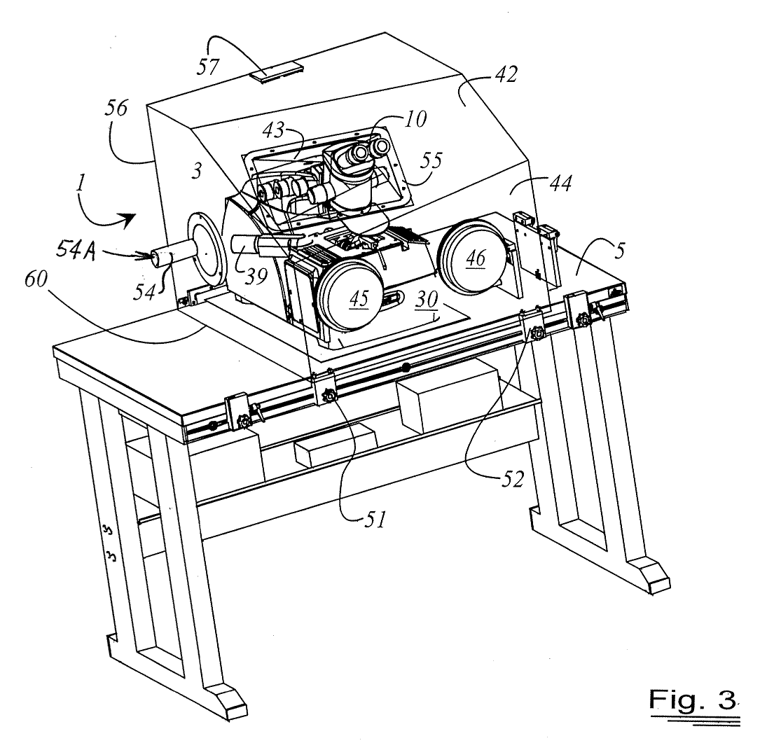

FIG. 3 is a perspective view of device 1 of FIG. 1 that is surrounded by a glovebox 42. Stereomicroscope 10 projects out of glovebox 42 via a movable but sealed bellows 43. A first and a second opening 45 and 46 are embodied on a front side 44 of glovebox 42. Openings 45 and 46 allow the installation of a flexible glove with which the user can operate the microtome or ultramicrotome in the interior of glovebox 42. Also conceivable are solutions that dispense with the glove and enclose the user's arm with elastic material. This is more advantageous, since the critical manipulations in glovebox 42 can be carried out more easily without a glove. The different configurations with which a user can gain access with his or her hands to the interior of glovebox 42 are evident from the Internet website of Plas●Labs, Inc. at www.plas-labs.com.

The manner in which device 1 functions may be described as follows. When cooling of cold chamber 30, and of the specimen holders present therein, begins, more than a liter of liquid coolant from reservoir vessel 35 is needed. The initially warm cold chamber 30 is thereby brought to operating temperature. The liquid coolant is liquid nitrogen. This means that approximately one cubic meter of gas (gaseous nitrogen) is produced, which is used to displace the atmosphere out of glovebox 42 and replace it with nitrogen gas. Only a slight overpressure occurs in this context, since a desired leakage rate exists because glovebox 42 is installed on table 5 without a seal. The flow of dry nitrogen gas is consequently sufficient to maintain this state.

It is possible to work in cold chamber 30 for many hours with no occurrence of ice contamination. Operation is independent of external laboratory conditions.

A laterally mounted airlock 50 serves for the introduction and removal of samples, knives, and accessories.

It is also conceivable to couple a cryosample holder of an electron microscope to glovebox 42 via airlock 50, so that transfer of the screen with the sections to the cryosample holder can likewise be performed in the dry nitrogen gas.

FIG. 3 shows glovebox 42, made of clear acrylic, placed on table 5 and fastened with multiple fastening elements 51 and 52. Sectioning apparatus 3 (a microtome or ultramicrotome) is located, with cold chamber 30 installed, in glovebox 42. Coolant hose 37 is guided into glovebox 42 via an elastic element 54 having a passthrough 54A, and is installed on cold chamber 30. Another passthrough, not shown, is preferably provided for cables running to sectioning apparatus 3. Stereomicroscope 10 is sealed via bellows 43 that is clamped in frame 55 of glovebox 42. The requisite range of motion of stereomicroscope 10 is not thereby restricted. Openings 45 and 46 are intended for manipulation. Elastic elements that rest against the user's arms, and close off openings 45, 46, are used. An overpressure valve 57 is provided, ensuring a slight overpressure in glovebox 42. Overpressure valve 57 is made up of a small plate that, in the event of overpressure, lifts up and uncovers an opening.

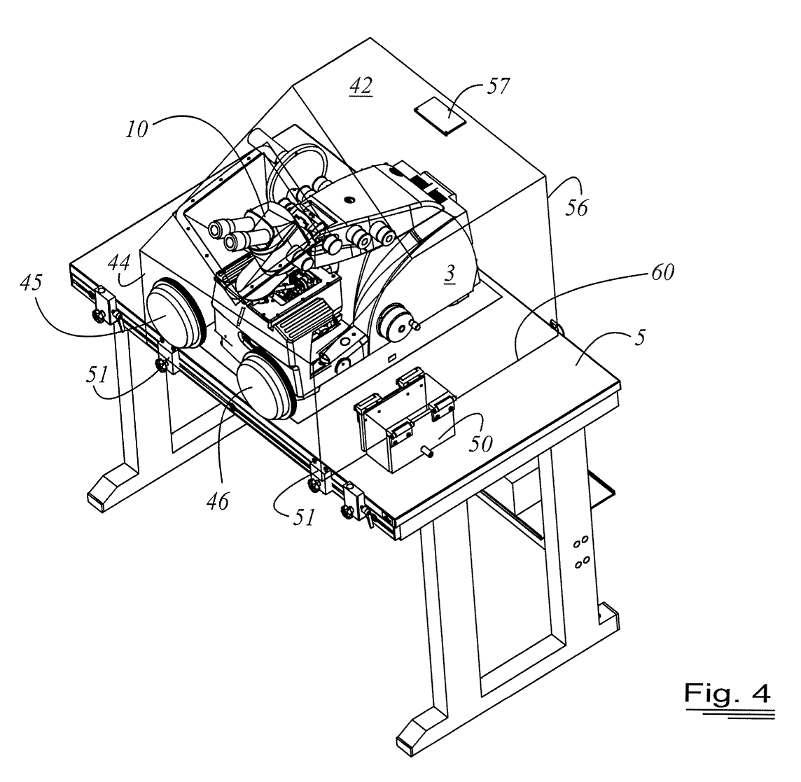

FIG. 4 is another view of device 1 of FIG. 3, showing airlock 50 with doors 14 on the outside and 15 on the inside. Glovebox 42 is fastened on back side 56 as well using additional fastening elements similar to fastening elements 51 and 52. Glovebox 42 possesses a rim 60 at which glovebox 42 contacts table 5. No seal is provided between rim 60 and table 5. Because no seal is provided, a specific leakage rate occurs so that a certain overpressure exists within glovebox 42. Coolant hose 37 is guided into glovebox 42 via elastic element 54.

What is claimed is:

1. A device for sectioning specimens comprising:
a sectioning apparatus for generating thin specimen pieces;
a table supporting the sectioning apparatus;
a cold chamber surrounding a region of the sectioning apparatus;
a glovebox on the table, the glovebox surrounding the sectioning apparatus and the cold chamber;
a reservoir vessel containing coolant; and
a coolant hose connecting the reservoir vessel to the cold chamber to enable coolant to flow from the reservoir vessel into the cold chamber,
wherein coolant evaporating out of the cold chamber fills the glovebox.

2. The device according to claim 1, wherein the glovebox includes a rim at which the glovebox contacts the table, and no seal is provided between the rim and the table.

3. The device according to claim 1, further comprising a plurality of fastening elements arranged to fasten the glovebox to the table.

4. The device according to claim 1, wherein the glovebox includes a passthrough for the coolant hose.

5. The device according to claim 1, wherein the sectioning apparatus includes a stereomicroscope for observation of the specimen, and a portion of the stereomicroscope is sealed by an elastic bellows between the interior of the glovebox and the laboratory environment.

6. The device according to one of claim 4, wherein the glovebox includes an elastic element for guiding the coolant hose into the glovebox, wherein the elastic element has the passthough for the coolant hose.

7. The device according to one of claim 1, wherein the glovebox includes a pair of openings covered by a flexible or elastic material such that a user can introduce his or her arm into the glovebox without causing a substantial leak.

8. The device according to claim 1, wherein the glovebox includes an overpressure valve that opens in response to relieve pressure within the glovebox.

9. The device according to claim 1, wherein the glovebox includes an airlock that permits introduction of tools and specimens into an interior of the glovebox while reducing an amount of moisture allowed to enter the interior of the glovebox during the introduction of tools and specimens.

10. The device according to claim 1, wherein the glovebox is equipped with an airlock that permits the introduction of a cryosample holder of an electron microscope into the interior of the glovebox, and allows the cryosample holder to be loaded with sample carriers and sections.
